(19) United States
(12) Patent Application Publication
(10) Pub. No.: US 2024/0254673 A1
(43) Pub. Date: Aug. 1, 2024

(54) METHOD AND DEVICE FOR DETERMINING SOILING

(71) Applicant: Henkel AG & Co. KGaA, Duesseldorf (DE)

(72) Inventors: Arnd Kessler, Monheim am Rhein (DE); Christian Nitsch, Duesseldorf (DE); Lars Zuechner, Langenfeld (DE); Georg Wawer, Wien (AT); Alexander Mueller, Monheim (DE); Wolfgang Wick, Dormagen (DE)

(73) Assignee: Henkel AG & Co. KGaA, Duesseldorf (DE)

(21) Appl. No.: 16/318,115

(22) PCT Filed: Jul. 11, 2017
(86) PCT No.: PCT/EP2017/067338

§ 371 Date: Jan. 15, 2019

(30) Foreign Application Priority Data
Jul. 15, 2016 (DE) 10 2016 212 978.3

(51) Int. Cl. D06F 34/18; C12Q 1/34; D06F 103/02

(52) U.S. Cl. CPC D06F 34/18; C12Q 1/34; D06F 2103/02

(57) ABSTRACT

The invention especially relates to a method, carried out by one or more devices, said method comprising: obtaining a measurement result of at least one biosensor (200) which result is representative of soiling (302) of a textile (304); determining, on the basis of the measurement result, at least one output variable that depends on the composition of the soling (302); and outputting or initiating output of the at least one output variable. The invention also relates to a device for carrying out said method, and to an associated computer program.

### FIELD OF THE INVENTION

The invention relates to methods and devices that can be used to determine soiling.

### BACKGROUND OF THE INVENTION

Soiling on textiles, such as items of clothing, curtains or bedding, is often difficult to identify. Soiling may not only impact the aesthetics of the textiles, but may also present a hygiene-related problem for the user of the textile.

Much soiling can be easily perceived by the naked eye, but the composition or origin of the soiling is often unclear to the user of the textile. In some cases, the user is for example not aware that the textile has even become soiled following a mishap. The soiling then only becomes apparent to the user at a later date, at which point the cause and the composition of the soiling is not clear to the user. Soiling having different compositions may appear very similar to the naked eye; for example, after a certain period of time, it may in particular no longer be possible to differentiate between blood stains and tomato stains with the naked eye.

In this case, it is desirable for the user to obtain an indication of the composition of the soiling. In particular, this allows hygiene-related problems with the textiles to be identified or ruled out. In addition, in most cases it is of interest to remove the soiling again by means of a cleaning process. Such cleaning processes may be made considerably easier, or may even be made possible in the first place, by indications of the composition of the soiling.

A method for optimizing a consumer apparatus is known for example from WO 01/96645 A2. To this end, a washing machine may comprise a sensor for detecting a property of a soiled item. This is intended to improve the cleaning of the item. Here, reference is made to pH sensors, conductivity sensors, water-hardness sensors, turbidity sensors, temperature sensors, calcium-ion sensors, and redox-potential sensors. It is however disadvantageous that, in some circumstances, in relation to soiling, sensors of this type do not provide sufficient information or reliable results regarding the stain.

### GENERAL DESCRIPTION OF SOME EXEMPLARY EMBODIMENTS OF THE INVENTION

Against the background of the identified prior art, the problem addressed by the invention is to at least partially reduce or prevent the above-described problems, i.e. to reliably provide indications on the composition of soiling on textiles. It is intended for it to be possible for the proposed methods and devices to be used in a domestic setting, in particular in a user-friendly manner.

According to a first aspect of the invention, a method is disclosed which is carried out by one or more devices, comprising: obtaining a measurement result from at least one biosensor that is representative of soiling of a textile; determining at least one output variable, which is dependent on the composition of the soiling, on the basis of the measurement result; and outputting or triggering the output of the at least one output variable.

According to a second aspect of the invention, a device is described that is designed or comprises corresponding means for carrying out and/or controlling a method according to the first aspect. Devices for the method according to the first aspect in particular are or comprise one or more devices according to the second aspect.

Soiling is in particular understood to mean an accumulation of foreign matter on the material of the textile or discoloration of the surface of the textile, in particular in the form of a stain, some dirt or a mark. For example, particles such as dust, traces of liquids, dyes or fatty residues are found on the surface. Furthermore, non-fixed textile dyes could also have been introduced into the textile, and the non-fixed textile dyes may dissolve out of the material during a cleaning process such as washing, for example. Soiling may also be understood to mean textile components, such as textile dyes, that have dissolved out.

A textile is understood in particular to be items of clothing, curtains, or bedding. Items of clothing and bedding for example include shirts, t-shirts, dresses, jackets, sweaters, trousers, blankets, covers, and pillow and duvet covers. The textiles may include various materials, for example natural fibers, chemical fibers, or even other materials such as leather.

It has been demonstrated that the measurement result from a biosensor can be used to reliably obtain information regarding the composition of soiling of a textile and to determine an output variable therefrom. It has been demonstrated that biosensors are advantageous, i.e. can be used particularly reliably, particularly for soiling that regularly occurs on textiles. As a result of using the one or more biosensors, a fingerprint of the soiling can be obtained.

A biosensor, or biochemical sensor, is in particular understood to be a sensor that uses an (in particular immobilized) biologically active system to generate the measurement result. In this process, a biochemical (for example enzymatic) reaction can be used. The biosensor may likewise comprise electronic components, such as signal amplifiers. A biosensor is in particular understood to be a biosensor in accordance with the IUPAC (International Union of Pure and Applied Chemistry) definition.

Here, the biosensor may for example be designed to detect or determine one or more different substances. To detect the substances to be determined, biosensors may use biological systems at different integration levels.

Preferably, the biologically active system of the biosensor interacts (indirectly or directly) with an analyte. The analyte may for example be the soiling or a sample thereof. Likewise, the analyte may include other substances, for example a solvent, for example water. By means of the interaction between the biological system and the analyte, physiochemical changes may in particular occur, such as changes in a layer thickness, a refractive index, light absorption or an electrical charge, to name but a few examples. These physiochemical changes are then determined so that a measurement result can be obtained. After the measurement process, the initial state of the biosensor is preferably re-established, such that it can be used again.

The measurement result being representative of soiling of a textile is in particular understood to mean that the soiling, and in particular the composition of the soiling, influences the measurement result at least in part. Since the composition of the soiling influences the resulting measurement result, an output variable that is dependent on the composition of the soiling can be determined from the measured variable.

In order to determine the output variable on the basis of the measurement result, it may for example be taken into account whether the measurement result has a particular value, and how high a value of the measurement result is. Likewise, however, the progression of the measurement result over time can also be taken into account, for example an increase and/or a decrease in the signal of the measurement result.

The measurement result obtained according to the first aspect is preferably representative of soiling on a surface of a textile.

The at least one output variable may in particular be one or more values that are representative of the chemical composition of the soiling, of the degree of soiling, and/or of the spatial distribution of the soiling. For example, the at least one output variable includes at least one value for the presence and/or the concentration of a chemical element or a chemical compound, the quantity of the soiling, or the surface area of the soiling.

By outputting or triggering the output of the at least one output variable, the user can thus be provided with information regarding the composition of the soiling of the textile that advantageously contributes to identifying the soiling. The user may for example be provided with information regarding the chemical composition or the presence of individual elements or compounds. In particular by means of classification, further information can be provided by means of the at least one output variable, for example whether the soiling contains any particular organic or inorganic components, such as dyes or lipids, polysaccharides, or proteins, and potentially the origin of the soiling. For example, the at least one output variable can give the user information as to whether the soiling possibly poses a hygiene-related issue.

The method according to the first aspect and the device according to the second aspect therefore in particular make it easier for the user to identify the composition or the origin of the soiling. If, for example, soiling cannot be identified with the naked eye, at least one output variable, which is dependent on the composition of the soiling, can be determined using the method and the device. The output variable can for example allow the user to differentiate between different compositions of soiling that have the same appearance to the naked eye.

Alternatively, it is likewise conceivable to in particular use another chemical sensor (i.e. a sensor for the qualitative and/or quantitative detection of chemical-physical properties of atoms, molecules, and/or ions in gases, liquids, and/or solids by generating electrical signals) instead of a biosensor. A chemical sensor is for example an electrochemical sensor. In this respect, alternative aspects are also intended to be disclosed, with a chemical sensor being provided instead of a biosensor. These alternative aspects can likewise be combined with the embodiments described here.

According to an embodiment of the method according the first aspect, the soiling is locatable or non-locatable soiling.

Locatable soiling is in particular locally delimited and in particular visible soiling, for example a stain. Non-locatable soiling is for example in particular invisible soiling of the textile, for example with dust, (skin) oil or sweat. Non-locatable soiling is also intended to be understood to be a non-fixed dye (or insufficiently fixed dye) of the textile or a dye that has dissolved out due to a washing process.

Usually, it should be possible to remove the soiling again by means of a cleaning process. In one embodiment of the method according to the first aspect, the at least one output variable comprises at least one parameter of a cleaning strategy of the textile. The user can thus be given a recommendation on a cleaning process that would be best for the present soiling.

For soiling, a conclusion can be drawn on parameters of the cleaning strategy using the measurement result, which is dependent on the composition of the soiling. Here, the at least one parameter of the cleaning strategy can be determined indirectly from another output variable; for example, an output variable representative of the composition of the soiling is first identified, and at least one parameter of the cleaning strategy is determined from said output variable representative of the composition of the soiling. At least one parameter can also be determined directly from the measurement result, for example by means of a classification using stored measurement results and cleaning strategies assigned to the classes.

In particular if the user of the textile cannot identify the soiling with the naked eye and it is therefore unclear how to remove the soiling, a recommendation on an optimal cleaning strategy can be provided using the method according to the first aspect and the device according to the second aspect. For example, it may be unclear to the user whether the soiling contains lipids or certain dyes which cannot be reliably removed using cleaning strategies that are usually applied. By means of the cleaning strategy that is determined as part of the method and is dependent on the composition of the soiling, a recommendation on a cleaning strategy adapted to the specific composition can be provided by identifying corresponding constituents of the soiling. As a result, the removal of the soiling can be significantly simplified and can be made much more reliable.

It is likewise conceivable for substances that have dissolved out of the material of the textile, for example non-fixed textile dyes, to be detected as soiling by means of the at least one output variable. This takes place in particular while the cleaning strategy is being carried out, such that the user is provided with a conclusion on the efficacy of the cleaning strategy. For example, the user can identify whether an excessively large quantity of textile dyes dissolves out of the material of the textile, which prompts the user to modify the cleaning strategy and render it gentler in terms of the textile dye. Equally, decolorization of a textile may be intended and a conclusion may be drawn on the degree of decolorization brought about by a cleaning strategy by means of the at least one output variable.

In particular, the at least one parameter of the cleaning strategy constitutes a type of cleaning agent, a quantity of cleaning agent, a cleaning temperature, a type of cleaning device, settings of a cleaning device or combinations thereof.

Cleaning agents are used in a domestic setting for cleaning different objects, for example. For example, a cleaning agent, e.g. a washing agent, is used in washing machines to clean textiles. A cleaning agent is, however, intended to be understood to also mean cleaning agent aids or cleaning additives, such as a bleaching additive, a softener, or starch. A cleaning agent may also be a liquid, a disperse system, for example a gel or a foam, or a solid, in particular a tablet, powder, or granulate.

A cleaning agent may for example comprise one or more components from the group of components including surfactants, alkalis, builders, optical brighteners, enzymes, bleaching agents, soil-release polymers, fillers, plasticizers, fragrances, dyes, care substances, acids, starch, isomalt, sugar, cellulose, cellulose derivatives, carboxymethylcellulose, polyetherimide, silicone derivatives, and/or polymethylimines.

A cleaning agent may also comprise one or more other components. These components include, but are not limited to, the group consisting of bleach activators, complexing agents, builders, electrolytes, non-aqueous solvents, pH adjusters, perfume carriers, fluorescing agents, hydrotropic substances, silicone oils, bentonites, anti-redeposition agents, shrinkage-preventing agents, crease-preventing agents, dye transfer inhibitors, anti-microbial active ingredients, germicides, fungicides, antioxidants, preservatives, corrosion inhibitors, antistatic agents, bittering agents, ironing aids, repellents and impregnating agents, anti-swelling and anti-slip agents, and/or UV absorbers.

The at least one parameter of the cleaning strategy can represent the type of cleaning agent and therefore can be indicative of the composition of the cleaning agent. If, for example, a certain proportion of dye is contained in the composition of the soiling, it may be recommended to the user that they should use certain bleaching additives. If, for example, there are certain levels of lipids in the composition of the soiling, the use of specific surfactants and/or lipases may be contained in the recommended cleaning strategy.

The at least one parameter can represent the quantity of cleaning agent and in particular can indicate an absolute quantity of the cleaning agent. Equally, a relative quantity of the cleaning agent can be indicated by the at least one parameter, for example based on the mass of the textiles to be cleaned and/or a liquor ratio, or a quantity of cleaning agent based on a water volume to be used for the cleaning. By means of the measurement result, which is dependent on the composition of the soiling, a type of cleaning agent and/or a quantity of cleaning agent can be determined which ensures the optimal removal of the soiling.

Using a parameter representative of the cleaning temperature, a temperature that is optimal for the determined composition of the soiling can be specified for removing the soiling, in particular in combination with a type of cleaning agent. Here, the cleaning temperature is high enough to ensure that the soiling is removed as fully as possible and is also kept low enough in order to save energy and to care for the textile.

A cleaning device is in particular understood to be a washing machine, in particular an automatic domestic washing machine.

The washing machine may have various different designs. A distinction is made between top-loading washing machines, in which the loading opening is on the top, and front-loading washing machines, in which a porthole on the front serves as the loading opening. An advantage of top-loading washing machines is that it is easier to construct the door seal and the drum can be supported on two sides by roller bearings, and a top-loading washing machine can also be positioned in very small spaces where there is not enough space to open a front door. By contrast, a front-loading washing machine provides space on top for e.g. a tumble dryer or for a worktop, and is therefore sometimes built into a kitchen unit instead of a floor unit. Top-loading washing machines are disadvantageous since they require a greater volume of water for washing laundry than front-loading washing machines.

American top-loading washing machines always have a rotating drum and mixing elements (agitators or discs), with the mixing elements being able to move in or counter to the rotational direction of the drum. The machines may comprise a suds circulator and injectors for the suds. In principle, a distinction is made between deep-fill and HE top-loading washing machines. Deep-fill top-loading washing machines operate at specified water levels, and therefore do not use any load detection. HE washing machines generally have load detection and control the quantities of water in accordance therewith. Generally, the machines do not have an integrated heater, but instead are connected to a hot water feed.

In the context of the present invention, a parameter of the cleaning strategy can specify a certain type of such a cleaning device. It is also conceivable for the parameter to specify cleaning strategies that are intended to be carried out manually at least in part, such as a handwash. The at least one parameter may also include settings for a cleaning device, for example a program for an automatic domestic washing machine or a sequence for such programs.

As a result, the at least one parameter of the cleaning strategy can make it considerably easier for the user to remove the soiling. In particular for soiling which cannot be identified with the naked eye, a cleaning strategy that is optimal both in terms of the cleaning but also in terms of energy consumption and caring for the material of the textile can be recommended using the method. For example, the recommendation on the type of cleaning agent and the settings for the cleaning device contains information as to whether the measurement result indicates a certain proportion of lipids in the soiling and therefore that corresponding degreasing components should be contained in the cleaning agent, or as to whether certain dyes are present in the soiling that can be tackled in a targeted manner by a specific type of cleaning agent and settings of the cleaning device.

The at least one parameter may include a recommendation on pre-treatment of the textile. Pre-treatment of this type for example involves manually or automatically applying a cleaning agent to the soiling, in particular for a specified contact time, the duration of which can also serve as a parameter of the cleaning strategy. The textile can then be cleaned in a cleaning device, for example an automatic domestic washing machine.

In particular, the method further comprises carrying out the cleaning strategy by means of a cleaning device.

In this process, the measurement result can be obtained before, during and/or after the cleaning strategy for the textile is carried out. By obtaining said result before the cleaning, the user can for example be given a recommendation of the cleaning strategy to be used before a cleaning treatment that is to be carried out.

If the measurement result is obtained during the cleaning, the cleaning can for example be carried out dynamically, i.e. a cleaning device can be adjusted during the cleaning to the at least one output variable that has just been determined, in particular by the output variable being continuously determined. For example, a washing machine adjusts the temperature or the quantity of cleaning agent during the washing program according to the determined output variable. Here, the measurement result can be obtained in particular from textile components, such as textile dyes, that have dissolved out of the textile.

When obtaining the measurement result after cleaning, the result or the efficacy of a cleaning strategy can be recorded and checked, for example.

The at least one output variable can be output to the user on a display, or a corresponding output can be triggered. The user can then carry out the cleaning strategy. Alternatively or additionally, in an embodiment of the method, the at least one output variable can be output to a cleaning device. For example, the at least one output variable may represent at least one parameter of a cleaning strategy that is output to the cleaning device, such that the cleaning device for example adopts the corresponding cleaning strategy as a preset and the user only has to start the cleaning device. It is equally conceivable for the cleaning device to automatically carry out the cleaning strategy when the at least one output variable is output. The cleaning device may for example have a dosing device for the cleaning agent, in order to automatically provide the type of cleaning agent and the quantity of cleaning agent in accordance with the recommended cleaning strategy. As a result, this makes the method more user-friendly.

In an embodiment of the method, determining the at least one output variable includes comparing the measurement result with comparative values. Corresponding comparative values can be stored in a database. The measurement result may be subject to a classification, with the at least one output variable being obtained or influenced by a result of the classification. A classification may for example be based on a comparison of the measurement result with a database of known measurement results.

The comparative values or a database provided for this purpose may in particular contain measurement results for typical soiling occurring in the fields of application of textiles. For example, in a domestic setting, measurement results for typical soiling such as different food residues, traces of beverages, grass or dyes can be drawn upon. The comparative values may include one or more values or value ranges. Furthermore, particular output variables may be assigned to the corresponding comparative values, for example at least one parameter of a cleaning strategy for removing the soiling.

In an embodiment of the method, the method further comprises determining the measurement result by means of the at least one biosensor. As already stated at the outset, a biosensor is in particular understood to be a sensor that uses a biologically active system to generate the measurement result.

The measurement result can in particular be determined by the biosensor being brought into contact with the soiled textile in the region of the soiling. In this respect, the soiling on the textile can constitute the analyte. For example, a sensitive region of the sensor is brought into contact with the soiling. It is, however, also possible for the soiling on the textile to be first be wetted with a solvent (for example water) and then to be brought into contact with the biosensor.

A solvent may for example be an aprotic (for example an aprotic nonpolar or an aprotic polar) solvent or a protic solvent.

It is also possible for the biosensor not to need to be brought into direct contact with the soiling on the textile. For example, the biosensor may also merely be brought into contact with the solvent, which was previously in contact with the soiling on the textile.

This may be particularly advantageous if a textile is already in the cleaning device and the dye has run due to the cleaning process. The soiling in the form of the non-fixed dye can then make possible a corresponding measurement result in a biosensor (which is for example freely movable in the cleaning device) that is representative of the soiling. In this case, the biosensor only needs to come into contact with the solvent in the form of the washing water.

In another embodiment of the method, the method further comprises: determining a soiling profile based at least in part on the output variable, in particular based on a plurality of determined output variables, the determination of the at least one output variable being based at least in part on the soiling profile.

By means of the at least one output variable, a soiling profile can thus be produced which is adjusted to the particular composition of the soiling. In particular, a plurality of output variables, in the sense of a history of determined output variables, can feed into a soiling profile, such that future determinations can be based on the soiling profile at least in part. Therefore, the determination of the at least one output variable can be designed to be adaptive, and is more precisely adjusted to the respective requirements by means of the soiling profile. The output variable can be determined more precisely in particular in terms of its dependence on the chemical composition of the soiling.

For example, a soiling profile can be produced with regard to frequently occurring compositions of soiling. For outputting at least one parameter of a cleaning strategy, in particular the type of cleaning agent and the type of cleaning device can also be taken into account in a soiling profile.

It is also conceivable for information on the efficacy of the cleaning strategy to be incorporated in the soiling profile. For example, a measurement result can be determined again after a cleaning process in order to determine the efficacy of the cleaning strategy. This can further optimize future cleaning strategies by means of the soiling profile.

Equally, the user can evaluate the at least one output variable, for example evaluate the efficacy of the cleaning strategy that is adopted in the soiling profile. Therefore, the determination of the output variable, in particular the cleaning strategy, can be personalized.

It is also possible for the information regarding the efficacy and/or the user's evaluation to be used for machine learning. This means that the soiling profile can for example be determined or optimized based on machine learning at least in part. Machine learning is in particular understood to mean that an artificial system (for example a device according to the second aspect or a system according to the third aspect) learns from examples, and can generalize said examples following the learning phase. This mean that it does not simply learn the examples by heart, but that patterns and regularities are recognized in the learning data. Various approaches can be taken for this purpose. For example, supervised learning, semi-supervised learning, unsupervised learning, reinforcement learning and/or active learning can be used. Supervised learning can for example be carried out by means of an artificial neural network (such as a recurrent neural network) or by means of a support vector machine. In addition, unsupervised learning can for example be carried out by means of an artificial neural network (for example an autoencoder). For example, in particular those measurement results that are obtained from or determined by the at least one biosensor and are representative of soiling of a textile and corresponding information on the efficacy of the cleaning and/or a corresponding user evaluation can then be used as learning data. For this purpose, measurement results from a plurality of users can also be used to further accelerate and improve the learning process.

Alternatively or additionally, it is conceivable for obtained and/or determined measurement results to be associated with other information, for example with the number and/or the age of the person in a household in order to produce a personalized usage profile, or for example with the seasons in order to produce a seasonal usage profile.

Using these measures, the reliability of the determination of the output variable can be further increased.

According to a preferred embodiment of the method according to the first aspect, the method further comprises determining property information for the textile, determining the at least one output variable additionally being based on the determined property information for the textile.

Property information for the textile may for example include information regarding the material of the textile. For example, the property information is indicative of the type of material of the textile. The type of material is in particular understood to mean the composition of at least part of the material of the textile. For example, the property information is indicative of natural fibers, chemical fibers, artificial fibers, or natural materials such as cotton, wool, silk, or leather in the textile. The type of material also has a significant impact on the optimal treatment of the textile, for example a cleaning treatment or an ironing process. Property information for the textile may likewise be representative of the color of the material of the textile.

Property information may alternatively or additionally include structural information regarding the textile. For example, the structural information is indicative of the material structure, material distribution, the material wear of the textile, or a combination thereof.

The material structure of the textile is in particular understood to mean the type and/or form of the textile, for example whether this is a woven fabric, a knitted fabric, a non-woven fabric, or a fibrous web. The corresponding structural information may in particular be characteristic of the way in which fibers are interlinked, such as how said fibers have been produced, for example by weaving or knitting, or may be characteristic of a non-woven fabric. An interlinking pattern or a thread-weaving pattern and thread interlacing may be included in the structural information here. Thread density, fiber thickness, fiber length, fiber fineness, and/or fiber orientation may in particular be included in the structural information. The material structure of the textile has a direct impact on the requirements for the treatment of the textile; for example, a non-woven fabric may have different requirements in terms of cleaning treatment than a knitted or woven structure.

The material distribution of the textile may for example make it possible to detect whether the textile comprises a blended fabric made up of different fiber types or fiber materials, and/or whether some regions of the textile are made of a different material. Here, the ratio of the different materials to one another, for example a density ratio, mass ratio, or surface-area ratio, can be determined. Furthermore, the type and number of connection points, such as seams, weld points, or bonding points, can be contained in the structural information.

Using the structural information that is indicative of material wear, it can in particular be ascertained whether pilling, tears, holes, worn patches, or other structural damage is found on the textile. In particular for pilling, which occurs when fibers become detached from the interlinked textile structure and are found on the surface of the textile in the form of bobbles, the type, shape, size or height, number, and/or distribution of the material wear can be determined.

By determining at least one treatment parameter based on the material wear, the treatment of the textile can be adjusted according to the material wear in order to prevent any further, increased material wear, or for example to remove pilling in order to restore the appearance of the textile.

Property information for the textile is preferably determined by means of a separate sensor, such as a structure sensor. Here, the structure sensor may for example determine information regarding the shape, texture, appearance, and/or composition of the textile and the material of the textile. The structure sensor may for example be an optical or acoustic sensor in this case.

According to a preferred embodiment of the method according to the first aspect, the at least one biosensor comprises at least one active biological system, at least one signal converter, and/or at least one electronic system.

A biosensor gets its selectivity and sensitivity in particular from the biological system used. The biological system is in particular an immobilized biological system. Immobilization is in particular understood to mean the spatial fixing of bacteria, cells, or enzymes in gel particles, capsules, or also in delimited reaction compartments. The biological system may also be referred to as a biological detection component or a bioreceptor. The biological system for example uses biomolecules of organisms or receptors modeled on biological systems in order to interact with the corresponding analyte.

For example, the biosensor is based on the direct spatial coupling of the biologically active system with the signal converter (or transducer) and the electronic system. By means of the interaction between the biological system and an analyte, physiochemical changes may occur. Said changes can be determined by means of the signal converter, such as optoelectrical sensors, amperometric or potentiometric electrodes, or special field-effect transistors (chemically sensitive field-effect transistor). The electronic system, which in particular may comprise an electronic amplifier, can amplify the signal from the signal converter such that it can be processed further.

A measurement result is thus determined by means of a biosensor in particular in the three following steps. First, the analyte is identified by the biological system of the biosensor. Second, the physiochemical changes that come about due to the interactions of the analyte with the biological system (for example a receptor) are converted into an electrical signal. Third, the signal is then amplified and/or processed. In this case, the signal conversion and the electronics can be combined for amplification and/or processing, e.g. in a microsensor system.

A signal converter of the biosensor may for example have an optical, piezoelectric, or electrochemical basis.

According to an embodiment of the method according to the first aspect, the biologically active system is based on antibodies, enzymes, organelles, microorganisms, nucleic acids, cells and/or tissues.

A biologically active system based on antibodies operates on the basis of antibody/antigen interaction. Here, the (specific) binding affinity of antibodies for a particular substance or an antigen is utilized. It has been demonstrated that binding of this type may also be reversible. A biosensor based on this principle advantageously has high specificity.

A biologically active system based on enzymes operates in particular on the basis of an enzymatic reaction. In this case, the analyte can be identified using different mechanisms. For example, the enzyme can convert the analyte into a detectable product. For example, enzyme inhibition or activation can be detected by the analyte. For example, a modification of the enzyme properties can be detected by means of the interaction with the analyte. A biologically active system based on enzymes is therefore particularly advantageous since a large number of reactions can be catalyzed, groups of analytes can be detected and different signal converters can be used. It is also advantageous for enzymes not to be consumed during the reaction, such that a corresponding sensor can easily be used multiple times and is suitable for long-term use.

Examples of enzymes are amylases, proteases (also referred to as peptidases), and/or lipases. Likewise, however, other hydrolases, esterases, and/or glycosidases can also be used.

A biologically active system based on nucleic acids operates in particular on the basis of nucleic-acid interactions, in particular using the principle of complementary base pairs (adenine:thymine and cytosine:guanine) in the DNA.

A biologically active system based on organelles operates in particular on the basis of the reaction of organelles with certain substances. It has already been possible to use systems and sensors of this type to detect water pollution. Sensors of this type can be used in the same way in relation to soiling on a textile.

A biologically active system based on cells has the advantage that a wide spectrum of analytes can be detected. In addition, corresponding sensors can be easily produced due to the cells, which are generally easy to handle. Sensors of this type also have a comparatively long activity period.

A biologically active system based on tissue is advantageously particularly easy to immobilize. Tissues also has comparatively high stability and activity. Lastly, corresponding sensors are also characterized by good availability at a low price. By comparison with enzyme-based biosensors, the extraction of corresponding enzymes is also dispensed with. However, compared with enzyme-based sensors, generally only lower specificity can be achieved.

According to an embodiment of the method according to the first aspect, the biologically active system is based on a lipase reaction, an amylase reaction, and/or a protease reaction.

By means of a biosensor of which the biologically active system is based on a lipase reaction, lipids can in particular be identified in the composition of the soiling. As a result, it can be determined that the composition of the soiling contains lipids and/or the proportion of lipids can be determined. If, for example, there are lipids or certain levels of lipids in the composition of the soiling, the use of specific surfactants and/or lipases may be contained in the recommended cleaning strategy.

By means of a biosensor of which the biologically active system is based on an amylase reaction, polysaccharides can in particular be identified in the composition of the soiling. As a result, it can in particular be determined that the composition of the soiling contains polysaccharides and/or the proportion thereof can be determined. If, for example, there are polysaccharides or certain levels of polysaccharides in the composition of the soiling, the use of specific surfactants may be contained in the recommended cleaning strategy.

By means of a biosensor of which the biologically active system is based on a protease reaction (also called a peptidase reaction), proteins can in particular be identified in the composition of the soiling. As a result, it can in particular be determined that the composition of the soiling contains proteins and/or the proportion thereof can be determined. If, for example, there are proteins or certain levels of proteins in the composition of the soiling, the use of specific surfactants or bleaching agents may be contained in the recommended cleaning strategy.

According to an embodiment of the method according to the first aspect, the at least one biosensor is designed to detect a lipid, a polysaccharide or a protein. As already stated, this can in particular be carried out by means of a biosensor having a biologically active system based on enzymes. However, biosensors having a different biologically active system are also used. Examples of lipids are fatty acids, triacylglycerides (fats and fatty oils), waxes, phospholipids, sphingolipids, lipopolysaccharides, and/or isoprenoids (steroids, carotinoids, etc.). Examples of polysaccharides are glycogen, starch (amylose and amylopectine), pectins, chitin, callose, and/or cellulose. The term “proteins” is in particular understood to mean proteins or protein substances. By means of one or more biosensors designed to detect substances of this type, a broad spectrum of typical instances of soiling on textiles and effective cleaning strategies can be proposed or carried out.

According to an embodiment of the method according to the first aspect, the at least one biosensor is a specific and/or non-specific biosensor. A specific biosensor is understood to be a biosensor that detects only a certain substance or substance group with sufficient reliability. A non-specific sensor is understood to be a sensor that detects a plurality of substances or substance groups. A specific sensor accordingly has the advantage that a single sensor can make a reliable declaration in relation to a certain substance in the composition. With a non-specific sensor, in some cases a plurality of sensors need to be provided in order to allow a certain substance to be detected.

According to an embodiment of the method according to the first aspect, a plurality of biosensors are provided for obtaining the measurement result. Advantageously, different substances in the composition of the soiling can be detected by a plurality of biosensors. If a plurality of biosensors are provided, they may all be specific or may all be non-specific, for example. It is likewise conceivable for some of the plurality of biosensors to be specific and some to be non-specific. In particular with regard to non-specific biosensors, it is therefore advantageous for a plurality of biosensors to be provided.

Alternatively or additionally, it is likewise conceivable for at least one biosensor to be provided with a plurality of sensitive (specific and/or non-specific) measurement regions. In this way, a more precise measurement result for the soiling can be obtained by means of just one biosensor.

In another embodiment of the method, at least one of the devices for carrying out the method is a portable apparatus. The portable apparatus may for example be a sensor device and may comprise the at least one biosensor. In particular, communication may be undertaken by means of a communication system between the portable apparatus, for example a smartphone, laptop, tablet, wearable, or a sensor device, and at least one other device, for example a cleaning device and/or a server. According to an embodiment, the device according to the second aspect comprises a communication interface. For example, the communication interface is designed for wired or wireless communication. For example, the communication interface is a network interface. The communication interface is preferably designed to communicate with a communication system. Examples of a communication system are a local network (LAN), a wide area network (WAN), a wireless network (for example in accordance with the IEEE-802.11 standard, the Bluetooth (LE) standard and/or the NFC standard), a wired network, a mobile network, a telephone network, and/or the Internet. A communication system may include communication with an external computer, for example over an Internet connection.

Other devices may be provided, for example a server and/or for example a part or component of a computer cloud, which provides data-processing resources dynamically for different users in a communication system. A computer cloud is in particular understood to mean a data-processing infrastructure in accordance with the definition from the National Institute for Standards and Technology (NIST) for the term “cloud computing.” One example of a computer cloud is a Microsoft Windows Azure platform.

In an alternative or additional embodiment, the measurement result can be determined by means of a biosensor, with the biosensor being arranged on a cleaning device. For example, the biosensor is arranged at least in part on the outer housing of a cleaning device and outside a cleaning container in which cleaning can be carried out. This means that the user always has access to the biosensor even if the cleaning device is in operation or is switched off. Therefore, a method according to the first aspect can be carried out at any time. In this case, the biosensor can be fastened to the cleaning device, for example using fastening means such as a screw and/or adhesive. Equally, the biosensor can also be arranged so as to be freely positionable on the cleaning device using positioning means, for example using a magnetic fixture.

In addition, the biosensor can be arranged inside the cleaning device at least in part, in particular in the region of a cleaning container of the cleaning device in which cleaning can be carried out. In particular, the biosensor is arranged in a position accessible to the user in this case. Here too, the biosensor can be fastened to the cleaning device, for example using fastening means, or can be freely positionable using positioning means. The biosensor is for example arranged on an opening in the cleaning container, in particular on a door. In a washing machine, the biosensor is for example arranged on the loading opening of the washing drum and/or on the seal of the loading opening.

The biosensor may equally be designed to be freely movable inside the cleaning container. In an embodiment, a sensor device is provided where the sensor device comprises: at least one biosensor for determining the measurement result, the device being designed to provide the measurement result while a cleaning strategy is being carried out in a cleaning container of a cleaning device. For example, the sensor device is designed to be arranged to be freely movable in a washing drum of a washing machine during a washing process. The sensor device may have a shape corresponding to the cleaning treatment, and for example may have a rounded, in particular spherical, shape. The determination device may also have corresponding impermeability and mechanical resistance such that a washing solution and aggressive cleaning agents do not impair the function of the sensor device. The sensor device can therefore provide measurement results for the soiling during a cleaning process in order to monitor the cleaning strategy. Determination of the measurement result can be provided both for soiling on the surface of a textile and/or for soiling such as dissolved textile components such as textile dyes, for example by analyzing the washing solution.

In particular, at least one biosensor that is located outside the cleaning container at least in part is combined with at least one biosensor that is located within the cleaning device or cleaning container at least in part and is in particular freely movable. A plurality of biosensors of the same type or different types may be provided. This means that the precision of the determination of the at least one output variable is improved since the biosensors can provide measurement results corresponding to different positions. In particular, at least one of the biosensors operates continuously at least periodically, such that measurement results are obtained at different points in time and in particular continuously during a cleaning treatment.

Equally, at least one biosensor may be arranged on a part of the cleaning agent packaging, and for example may be arranged to be integrated in a closure cap or arranged on a closure flap, in particular by being fitted thereon. This means that the biosensor is movable freely and independently of a cleaning device and can be used by the user in a simple manner on soiling on a textile. The biosensor can then equally be used in conjunction with a plurality of different cleaning devices.

According to the second aspect of the invention, a device is described that comprises at least one processor and at least one memory comprising computer program code, wherein the at least one memory and the computer program code are designed to carry out and/or control at least one method according to the first aspect using the at least one processor. A processor is for example intended to be understood to be a control unit, a microprocessor, a microcontrol unit such as a microcontroller, a digital signal processor (DSP), an application-specific integrated circuit (ASIC) or a field programmable gate array (FPGA).

For example, an exemplary device further comprises means for storing information, such a program memory and/or a main memory. For example, an exemplary device according to the invention further comprises means for respectively receiving and/or transmitting information over a network, such as a network interface. For example, exemplary devices according to the invention are and/or can be interconnected via one or more networks.

An exemplary device according to the second aspect for example is or comprises a data-processing system set up using software and/or hardware in order to execute the respective steps of an exemplary method according to the second aspect. Examples of a data-processing system are a computer, a desktop computer, a server, a thin client and/or a portable computer (mobile device), such as a laptop computer, a tablet computer, a wearable, a personal digital assistant or a smartphone.

According to the second aspect of the invention, a computer program is also described that comprises program instructions that prompt a processor to execute and/or control a method according to the first aspect when the computer program runs on the processor. An exemplary program according to the invention may be stored in or on a computer-readable memory medium which contains one or more programs.

According to the second aspect of the invention, a computer-readable memory medium is also described which contains a computer program according to the second aspect. A computer-readable memory medium may be designed e.g. as a magnetic, electrical, electromagnetic, optical and/or different memory medium. A computer-readable memory medium of this type is preferably a physical object (i.e. “tangible”); for example, it is designed as a data carrier device.

A data carrier device is for example portable or permanently installed in a device. Examples of a data carrier device of this type are volatile or non-volatile memories with random access (RAM), such as NOR flash memories, or with sequential access, such as NAND flash memories, and/or memories with read-only access (ROM) or read/write access. Computer-readable is for example intended to be understood to mean that the memory medium can be read and/or written by a computer or a data-processing system, for example by a processor.

According to a third aspect of the invention, a system is also described that comprises a plurality of devices, in particular a portable apparatus (in particular a sensor device) and a cleaning device, which together carry out a method according to the first aspect.

An exemplary system according to the third aspect comprises an exemplary cleaning device and additionally another device, for example a portable apparatus or a server for carrying out an exemplary method according to the first aspect.

The exemplary embodiments of the present invention described above in this description are also intended to be understood to be disclosed in any combination with one another. In particular, exemplary embodiments are intended to be understood to be disclosed in relation to different aspects.

In particular, by way of the preceding or following description of method steps according to preferred embodiments of a method, corresponding means for carrying out the method steps are also intended to be disclosed by preferred embodiments of a device. Likewise, the disclosure of means of a device for carrying out a method step is also intended to disclose the corresponding method step.

Further advantageous, exemplary embodiments of the invention are found in the following detailed description of some exemplary embodiments of the present invention, in particular in conjunction with the drawings. The drawings are however only provided for illustrative purposes, and do not serve to define the scope of protection of the invention. The drawings are not to scale and are merely intended to provide an example of the general concept of the present invention. In particular, features contained in the drawings are not in any way intended to be considered as necessary components of the present invention.

### BRIEF DESCRIPTION OF THE DRAWINGS

In the drawings:

FIG. 1 is a flow diagram of an embodiment of a method;

FIG. 2 is a schematic view of an embodiment of a biosensor;

FIG. 3 is a schematic view of an embodiment of a device;

FIG. 4 is a block diagram of an embodiment of a device; and

FIG. 5 shows different embodiments of a memory medium.

### DETAILED DESCRIPTION OF SOME EXEMPLARY EMBODIMENTS OF THE INVENTION

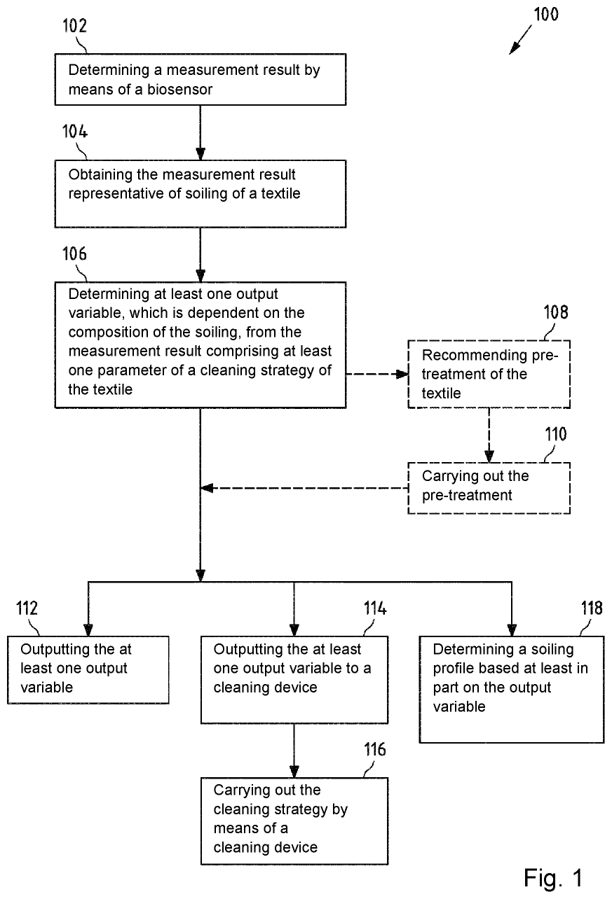

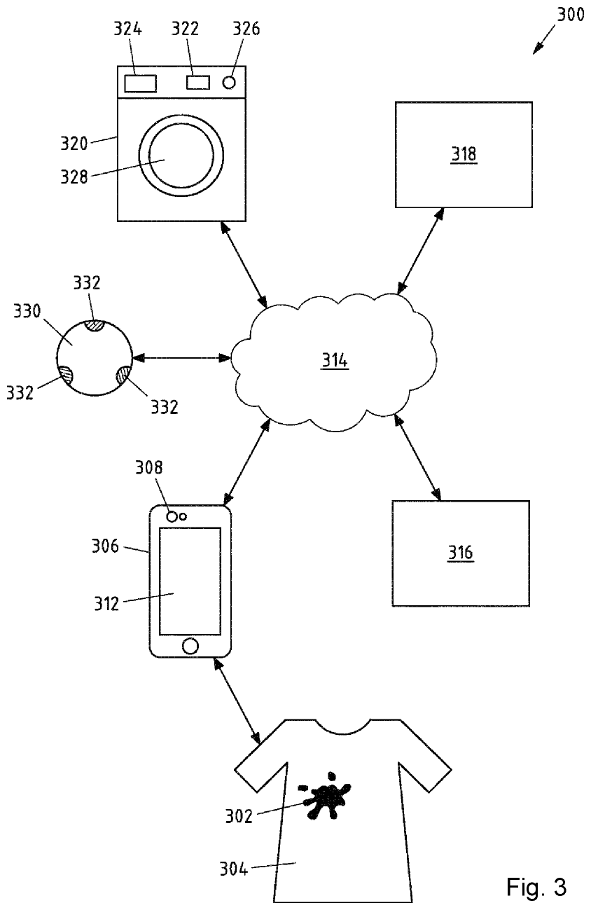

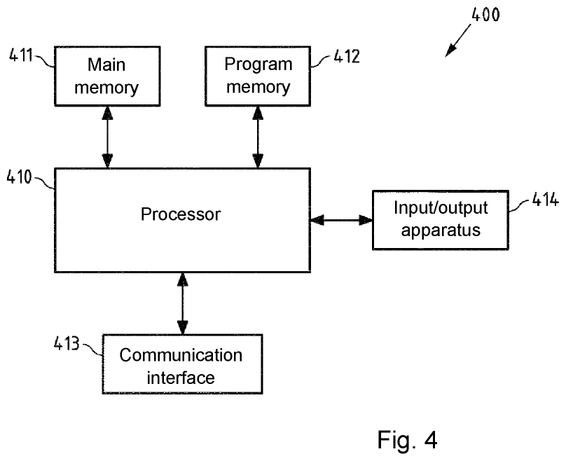

FIG. 1 is a flow diagram 100 of an embodiment of a method according to the first aspect, which is carried out by a device, for example one of the devices from FIGS. 3 and 4.

In action 102, a measurement result is determined by a biosensor, the measurement result being representative of soiling on a textile (for example soiling 302 on the textile 304 from FIG. 3).

This measurement result is obtained in action 104. Since the measurement result is dependent on the chemical composition of the soiling on the textile, in action 106 at least one output variable, which is dependent on the composition of the soiling, can be determined from the measurement result. Here, at least one parameter of a cleaning strategy of the textile is determined on the basis of the chemical composition of the soiling, the cleaning strategy constituting a recommendation on optimally cleaning the soiling from the textile.

In addition, in action 108, a recommendation can be made on pre-treatment of the textile. For certain compositions of the soiling, corresponding pretreatment may be required which is for example carried out by another device in action 110.

In action 112, the at least one output variable is prompted to be output, for example output to a display element, with information regarding the composition of the soiling and at least one parameter of the cleaning strategy in particular being displayed to the user. The user can carry out cleaning of the textile on the basis of the displayed information or recommendation.

Additionally or alternatively, in action 114 at least one output variable, in particular the at least one parameter of the cleaning strategy, can be output to a cleaning device. The output parameters of the cleaning strategy are used in action 116 to carry out cleaning by means of the cleaning device.

Additionally, in action 118 a soiling profile can be determined which is based at least in part on the output variable. Therefore, the determination of the at least one output variable can be designed to be adaptive, and is more precisely adjusted to the respective requirements by means of the soiling profile.

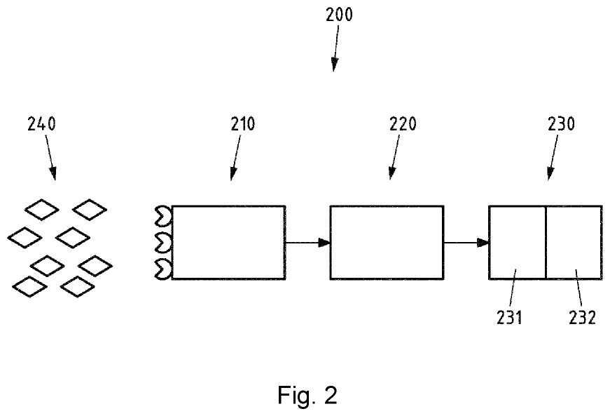

FIG. 2 is a schematic view of an embodiment of a biosensor 200. The biosensor comprises a biologically active, immobilized system 210, a signal converter (transducer) 220 and an electronic system 230.

The biologically active, immobilized system 210 comprises bioreceptors, which for example may comprise antibodies, enzymes, organelles, microorganisms, nucleic acids, cells and/or tissues. A lipase reaction, an amylase reaction, and/or a protease reaction can be carried out by the bioreceptors in the presence of the appropriate analyte 240.

The analyte 240 may for example be the soiling on the textile. In this case, the analyte may also comprise a solvent. Likewise, the analyte may be present independently of the textile, and may for example include a solvent that comprises parts of the soiling. For example, the analyte may be washing water from a cleaning process of a cleaning device which contains traces of the soiling.

The signal converter 220 is for example optically, piezoelectrically, or electrochemically based and for example comprises an optoelectrical sensor, amperometric or potentiometric electrodes, or special field-effect transistors.

The electronic system 230 for example comprises an electronic amplifier 231, and/or an evaluation unit 232. The measurement result can for example be provided by the evaluation unit. It is likewise conceivable that an output variable has already been determined by the evaluation unit 231. This may, however, also be carried out by a different data-processing system (for example a device 316 from FIG. 3).

FIG. 3 shows an embodiment of a device 300 according to the second aspect and a system according to the third aspect. The device 300 is designed to or comprises corresponding means to carry out and/or control a method according to the first aspect.

In particular, the device 300 makes it possible to identify a composition of soiling 302 on a textile 304 and/or gives a recommendation on a cleaning strategy for removing the soiling 302 from the textile 304.

Using a portable apparatus, in this case a sensor device 306, a measurement result is first representative of soiling 302. The biosensor 308 is used for this purpose. The sensor device 306 also has a display element 312.

The determined measurement result is received by a communication system 314. A data-processing system 316 designed to determine output variables that are dependent on the composition of the soiling 302 from the measurement result is connected to the communication system 314.

Determining the output variables includes comparing the measurement result with comparative values in this case. The comparative values are stored in a database 318, which is likewise connected to the communication system 314. The comparative values in the database 318 in particular contain measurement results for soiling typically occurring in a domestic setting. In addition, the database 318 contains data assigned to the comparative values in the form of a chemical composition and/or parameters relating to a recommended cleaning strategy that is optimal for the corresponding composition. The devices 308, 3016 and/or 318 may, however, also be implemented by one device.

The output variables include parameters of a cleaning strategy of this type, the parameters specifying a type of cleaning agent, a quantity of cleaning agent, a cleaning temperature, a type of cleaning device, and/or settings of a cleaning device 320. These output variables may for example be displayed on the display element 312 of the sensor device 306 and can thus be made available to the user. The user is therefore provided with a recommendation on a cleaning strategy that is optimal for the specific soiling 302.

The cleaning device 320 is also in communication with the communication system 314, by means of which the output variables are output to the cleaning device 320. The cleaning device 320 comprises a display element 322, which in particular can display the output variables. In addition, the cleaning device 320 comprises a dosing device 324 for the cleaning agent. In this case, the dosing device 324 may provide a cleaning agent in accordance with the parameters of the cleaning strategy in relation to the type of cleaning agent and/or the quantity of cleaning agent, or may check whether the dosing device 324 has been filled with the cleaning agent in accordance with the recommended cleaning strategy.

In addition, the cleaning device 320 comprises a control element 326 which allows the cleaning device 320 to be controlled by a user. Here, the cleaning device 320 adopts the parameters of the cleaning strategy as a preset. The user then has the choice either to follow the recommendation on the cleaning strategy and simply start the cleaning device 320 by means of the control element 326, or to manually set the cleaning device 320 themselves by means of the control element 326. The cleaning is carried out in a cleaning container 328, in this case a washing drum.

In addition, FIG. 3 shows an alternative sensor device 330. The sensor device 330 comprises a plurality of sensors 332. The sensor device has a spherical shape and is designed to be arranged in the cleaning container 328 while cleaning is being carried out. In this case, the sensor device 330 is freely movable and is resistant to the action of the washing solution in the cleaning container 328. The sensor device 330 can therefore provide measurement results for the soiling 302 during a cleaning process in order to monitor the cleaning strategy. The sensor device 330 can also determine measurement results for soluble, non-fixed textile dyes in the washing solution. Therefore, the corresponding textile dyes dissolving out of the textile 304 can be monitored.

FIG. 4 is a block diagram of an embodiment of a device 400, which in particular can carry out an exemplary method according to the first aspect. The device 400 is for example a device according to the second aspect or a system according to the third aspect, or a part thereof.

In this respect, the device 400 may for example be a computer, a desktop computer, a server, a thin client or a portable computer (mobile device), such as a laptop computer, a tablet computer, a wearable, a personal digital assistant (PDA) or a smartphone. The device may for example perform the function of a server or a client.

The processor 410 of the device 400 is in particular designed as a microprocessor, a microcontrol unit, a microcontroller, a digital signal processor (DSP), an application-specific integrated circuit (ASIC) or a field programmable gate array (FPGA).

The processor 410 executes program instructions that are stored in the program memory 412, and for example stores intermediate results or the like in a working memory or main memory 411. For example, the program memory 412 is a non-volatile memory such as a flash memory, a magnetic memory, an EEPROM memory (electrically erasable, programmable read-only memory), and/or an optical memory. The main memory 411 is for example a volatile or non-volatile memory, in particular a memory with random access (RAM) such as a static RAM memory (SRAM), a dynamic RAM memory (DRAM), a ferroelectric RAM memory (FeRAM), and/or a magnetic RAM memory (MRAM).

The program memory 412 is preferably a local data carrier that is permanently connected to the device 400. Data carriers that are permanently connected to the device 400 are for example hard drives that are integrated in the device 400. Alternatively, the data carrier may for example also be a data carrier that can be detachably connected to the device 400, such as a memory stick, a removable storage device, a portable hard drive, a CD, a DVD, and/or a floppy disk.

The program memory 412 for example contains the operating system of the device 400, which is loaded in the main memory 411 at least in part and is executed by the processor 410 when the device 400 is started up. In particular, when starting up the device 400, at least part of the core of the operating system is loaded in the main memory 411 and executed by the processor 410. The operating system of the device 400 is for example a Windows, UNIX, Linux, Android, Apple IOS and/or MAC operating system.

The operating system in particular allows the device 400 to be used for data processing. It for example manages operating equipment such as the main memory 411 and the program memory 412, the network interface 413, and the input and output apparatus 414, inter alia provides basic functions by means of programming interfaces of other programs, and controls the execution of programs.

The processor 410 controls the communication interface 413, which for example may be a network interface and may be designed as a network card, network module, and/or modem. The communication interface 413 is in particular designed to establish a connection between the device 400 and other devices, in particular via a (wireless) communication system, for example a network, and to communicate therewith. The communication interface 413 may for example receive data (via the communication system) and forward said data to the processor 410, and/or receive and transmit data from the processor 410 (via the communication system). Examples of a communication system are a local network (LAN), a wide area network (WAN), a wireless network (for example in accordance with the IEEE-802.11 standard, the Bluetooth (LE) standard and/or the NFC standard), a wired network, a mobile network, a telephone network, and/or the Internet.

Furthermore, the processor 410 can control at least one input/output apparatus 414. The input/output apparatus 414 is for example a keyboard, a mouse, a microphone, a touch-sensitive display unit, a speaker, a read apparatus, a drive, and/or a camera. The input/output apparatus 414 may for example receive user inputs and forward said inputs to the processor 410, and/or receive and output information for the user of the processor 410.

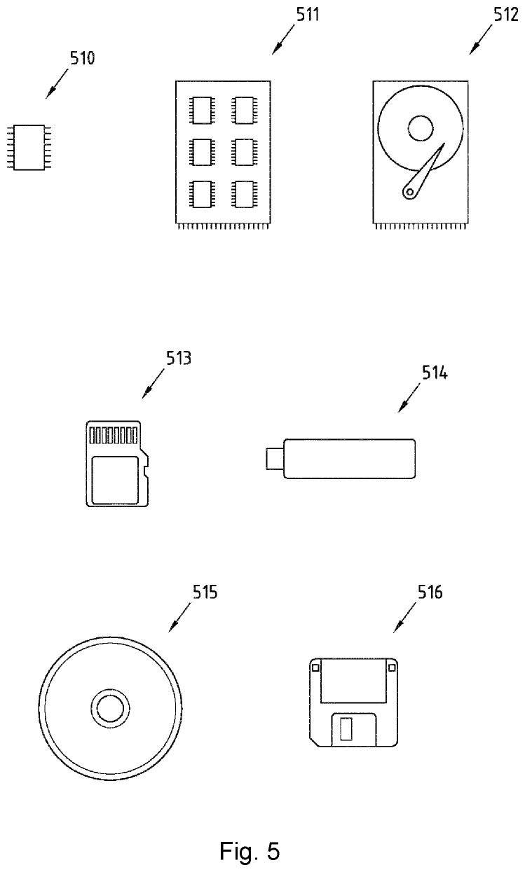

FIG. 5 lastly shows different embodiments of memory media on which an embodiment of a computer program according to the invention can be stored. The memory medium may for example be a magnetic, electrical, optical and/or different memory medium. The memory medium may for example be part of a processor (e.g. the processor 410 in FIG. 4), for example a (non-volatile or volatile) program memory of the processor, or a part thereof (such as the program memory 412 in FIG. 4). Embodiments of a memory medium are a flash memory 510, an SSD hard drive 511, a magnetic hard drive 512, a memory card 513, a memory stick 514 (e.g. a USB stick), a CD-ROM or DVD 515, or a floppy disk 516.

The embodiments of the present invention described in this specification and the optional features and properties set out in this regard in each case are also intended to be understood to be disclosed in any combination with one another. In particular, unless explicitly stated otherwise, the description of a feature included in an embodiment should not be understood in the present case such that the feature is indispensable or essential for the function of the embodiment. The sequence of method steps set out in this specification in the individual flow diagrams is not compulsory, and alternative sequences of the method steps are conceivable. The method steps can be implemented in different ways, and therefore implementation in software (by means of program instructions), hardware, or a combination of both are conceivable for implementing the method steps.

Terms used in the claims such as “include,” “comprise,” “contain” and the like do not exclude additional elements or steps. The wording “at least in part” covers both “partly” and also “completely.” The wording “and/or” is intended to be understood such that both the alternative and the combination are intended to be disclosed, i.e. “A and/or B” means “(A) or (B)” or “(A and B).” The use of the indefinite article does not exclude a plurality. A single device can carry out the functions of a plurality of units or devices mentioned in the claims. Reference signs stated in the claims should not be considered to limit the means and steps used.

1. A method carried out by one or more devices, comprising:
obtaining a measurement result from at least one biosensor (200) that is representative of soiling (302) of a textile (304);
determining at least one output variable, which is dependent on the composition of the soiling (302), on the basis of the measurement result; and
outputting or triggering the output of the at least one output variable.

2. The method according to claim 1, wherein the soiling (302) is locatable or non-locatable soiling.

3. The method according to claim 1 or 2, wherein the at least one output variable comprises at least one parameter of a cleaning strategy of the textile (304).

4. The method according to claim 3, wherein the at least one parameter of the cleaning strategy constitutes a type of cleaning agent, a quantity of cleaning agent, a cleaning temperature, a type of cleaning device, settings of a cleaning device (320), or combinations thereof.

5. The method according to claim 3 or 4, wherein the at least one parameter of the cleaning strategy includes a recommendation on pre-treatment of the soiling (204, 302).

6. The method according to one of claims 3 to 5, the method further comprising:
carrying out the cleaning strategy by means of a cleaning device (320).

7. The method according to one of claims 1 to 6, wherein the at least one output variable is output to a cleaning device (320).

8. The method according to one of claims 1 to 7, wherein determining the at least one output variable includes comparing the measurement result with comparative values.

9. The method according to one of claims 1 to 8, the method further comprising:
determining the measurement result by means of the at least one biosensor (200).

10. The method according to one of claims 1 to 9, the method further comprising:
determining a soiling profile based at least in part on the output variable, in particular based on a plurality of determined output variables,
the determination of the at least one output variable being based at least in part on the soiling profile.

11. The method according to one of claims 1 to 10, the method further comprising:
determining property information for the textile (304), determining the at least one output variable additionally being based on the determined property information for the textile (304).

12. The method according to one of claims 1 to 11, wherein the at least one biosensor (200) comprises at least one biologically active system (210), at least one signal converter (220), and/or at least one electronic system (230).

13. The method according to claim 12, wherein the biologically active system (210) is based on antibodies, enzymes, organelles, microorganisms, nucleic acids, cells, and/or tissues.

14. The method according to claim 12 or 13, wherein the biologically active system (210) is based on a lipase reaction, an amylase reaction, and/or a protease reaction.

15. The method according to one of claims 1 to 14, wherein the at least one biosensor (200) is designed to detect a lipid, a polysaccharide, or a protein.

16. The method according to one of claims 1 to 15, wherein the at least one biosensor (200) is a specific and/or non-specific biosensor.

17. The method according to one of claims 1 to 16, wherein a plurality of biosensors are provided for obtaining the measurement result.

18. The method according to one of claims 1 to 17, wherein at least one of the devices for carrying out the method is a portable apparatus (306, 322).

19. A device that is designed or comprises corresponding means for carrying out and/or controlling a method according to one of claims 1 to 18.

20. A device comprising at least one processor (410) and at least one memory (411, 412) comprising computer program code, wherein the at least one memory (411, 412) and the computer program code are designed to carry out and/or control at least one method according to one of claims 1 to 18 using the at least one processor (410).

21. A computer program comprising program instructions that prompt a processor (410) to execute and/or control a method according to one of claims 1 to 18 when the computer program runs on the processor (410).

22. A computer-readable memory medium which contains a computer program according to claim 21.

23. A system comprising:
a plurality of devices (306, 322, 314, 316, 318, 320), in particular at least one portable apparatus (306, 322) and a cleaning device (320), which together carry out a method according to one of claims 1 to 18.
